(19) United States
(12) Patent Application Publication
(10) Pub. No.: US 2021/0085904 A1
(43) Pub. Date: Mar. 25, 2021

(54) ORAL APPLIANCE FOR INCREASING OXYGENATION CAPACITY

(71) Applicant: Yesenia Garcia, Pearland, TX (US)

(72) Inventor: Yesenia Garcia, Pearland, TX (US)

(21) Appl. No.: 17/031,329

(22) Filed: Sep. 24, 2020

Related U.S. Application Data
(60) Provisional application No. 62/905,027, filed on Sep. 24, 2019.

(51) Int. Cl. A61M 16/04; A61M 16/06

(52) U.S. Cl. CPC A61M 16/0493; A61M 2205/0205; A61M 16/0627; A61M 16/047

(57) ABSTRACT

An oral appliance for increasing oxygenation including: an upper component configured to fit onto at least a portion of upper teeth of a user and including an upper locking mechanism; and a lower component configured to fit onto at least a portion of lower teeth of the user and including a lower locking mechanism configured to engage the upper locking mechanism in at least one position of the lower component relative to the upper component corresponding to at least one protrusion position of a mandible of the user of 50% of maximum protrusion to set an oxygenation capacity of the user; wherein the upper locking mechanism and the lower locking mechanism are configured to be lockable or unlockable or both by a pressure of a tongue the user or the mandible or both.

### CROSS-REFERENCE TO RELATED APPLICATIONS

This application claims priority to U.S. Provisional Application Serial No. 62/905,027, filed Sep. 24, 2019, the entire contents of which are incorporated herein by reference.

### STATEMENT OF FEDERALLY FUNDED RESEARCH

Not applicable.

### TECHNICAL FIELD OF THE INVENTION

The present invention relates in general to the field of oral appliances, and more particularly, to methods and systems for increasing oxygenation with an oral appliance for athletes.

### BACKGROUND OF THE INVENTION

Researchers have found that obstructive sleep apnea (OSA) is a common problem in the United States. Medicine and dentistry have addressed OSA with a variety of therapies. In the past, nightguards built with the teeth in centric relation (CR) were used to treat OSA because CR is the most relaxed and the most repetitive and predictable form of bite position for patients. However, research has shown that when the muscles relax, the tongue collapses into the airway and the patient's apnea worsens. Today, nightguards in CR are contraindicated for patients with OSA.

One of the accepted therapies for mild to moderate OSA is the use of an oral appliance that advances the mandible to 50% or more of the maximum mandible protrusion. This moves the tongue away from the pharynx and increases the volume of air that can go to the lungs. This treatment has been found to decrease the risk of several conditions and disorders including high blood pressure, high cholesterol, obesity, depression, migraines, headaches, temporomandibular joint pathologies, and bruxism.

One of the characteristics found in people with OSA is a thick neck. When the neck muscles are thick, the airway obstruction increases. When certain oral appliances are used to treat these OSA patients, their oxygenation has been found to be increased.

There is, in addition, a need for an oral appliance to increase oxygenation for athletes performing in sports activities.

### SUMMARY OF THE INVENTION

In some embodiments of the disclosure, an oral appliance for increasing oxygenation of an athlete is disclosed as including an upper component configured to fit onto at least a portion of upper teeth of a user and including an upper locking mechanism; and a lower component configured to fit onto at least a portion of lower teeth of the user and including a lower locking mechanism configured to engage the upper locking mechanism in at least one position of the lower component relative to the upper component corresponding to at least one protrusion position of a mandible of the user of 50% of a maximum protrusion distance of the mandible to set an oxygenation capacity of the user; wherein the upper locking mechanism and the lower locking mechanism are configured to be lockable or unlockable or both by a pressure of a tongue the user or the mandible or both. In one aspect, the upper locking mechanism and the lower locking mechanism each comprise at least one stub and recess configured to engage each other when a locking pressure is applied with the tongue or the mandible or both; the upper locking mechanism and the lower locking mechanism each comprise at least one stub and recess configured to disengage each other when an unlocking pressure is applied with the tongue or the mandible or both; the upper locking mechanism and the lower locking mechanism are configured to be lockable or unlockable or both during use of the oral appliance; the upper locking mechanism and the lower locking mechanism have at least 3 positions; the upper locking mechanism and the lower locking mechanism are configured to accommodate an underbite or an overbite of the user; or the upper locking mechanism and the lower locking mechanism advance from a centric relation at least 1, 2, 3, 4, 5, 6, 7, 8, 9, or 10 mm. In another aspect, the oral appliance further comprises at least one coupling component that couples the upper component and the lower component. In another aspect, the oral appliance further comprises a strap for attachment to a helmet, facemask, or other face or head protecting device. In another aspect, the at least one position of the lower component relative to the upper component corresponding to at least one protrusion position of the mandible comprises protrusion positions in a range of 0% to 100% of the maximum protrusion distance to allow adjustment of the oxygenation capacity of the user. In another aspect, the oral appliance further comprises at least one of the upper component, the lower component, the upper locking mechanism, or the lower locking mechanism comprises plastic, rubber, or acrylic that can be molded to the upper, lower, or both the upper and lower teeth. In another aspect, the oral appliance is created in a predetermined size. In another aspect, the upper locking mechanism, the lower locking mechanism, or both, is oriented vertically or partially vertically, but not horizontally.

In some embodiments of the disclosure, a kit is disclosed as including at least one oral appliance for increasing oxygenation of an athlete including: an upper component configured to fit onto at least a portion of upper teeth of a user and including an upper locking mechanism; and a lower component configured to fit onto at least a portion of lower teeth of the user and including a lower locking mechanism configured to engage the upper locking mechanism in at least one position of the lower component relative to the upper component corresponding to at least one protrusion position of a mandible of the user of 50% of a maximum protrusion distance of the mandible to set an oxygenation capacity of the user; wherein the upper locking mechanism and the lower locking mechanism are configured to be lockable or unlockable or both by a pressure of a tongue the user or the mandible or both. In one aspect, the upper locking mechanism and the lower locking mechanism each comprise at least one stub and recess configured to engage each other when a locking pressure is applied with the tongue or the mandible or both; the upper locking mechanism and the lower locking mechanism each comprise at least one stub and recess configured to disengage each other when an unlocking pressure is applied with the tongue or the mandible or both; the upper locking mechanism and the lower locking mechanism are configured to be lockable or unlockable or both during use of the oral appliance; the upper locking mechanism and the lower locking mechanism have at least 3 positions; the upper locking mechanism and the lower locking mechanism are configured to accommodate an underbite or an overbite of the user; or the upper locking mechanism and the lower locking mechanism advance from a centric relation at least 1, 2, 3, 4, 5, 6, 7, 8, 9, or 10 mm. In another aspect, the oral appliance further comprises at least one coupling component that couples the upper component and the lower component. In another aspect, the oral appliance further comprises a strap for attachment to a helmet, facemask, or other face or head protecting device. In another aspect, the at least one position of the lower component relative to the upper component corresponding to at least one protrusion position of the mandible comprises protrusion positions in a range of 0% to 100% of the maximum protrusion distance to allow adjustment of the oxygenation capacity of the user. In another aspect, the oral appliance further comprises at least one of the upper component, the lower component, the upper locking mechanism, or the lower locking mechanism comprises plastic, rubber, or acrylic that can be molded to the upper, lower, or both the upper and lower teeth. In another aspect, the oral appliance is created in a predetermined size. In another aspect, the upper locking mechanism, the lower locking mechanism, or both, is oriented vertically or partially vertically, but not horizontally.

In some embodiments of the disclosure, a method of increasing oxygenation is disclosed as including providing a user for whom increased oxygenation is desirable; determining or making an oral appliance of appropriate size for the user, wherein the oral appliance includes: an upper component configured to fit onto at least a portion of upper teeth of the user and including an upper locking mechanism; and a lower component configured to fit onto at least a portion of lower teeth of the user and including a lower locking mechanism configured to engage the upper locking mechanism in at least one position of the lower component relative to the upper component corresponding to at least one protrusion position of a mandible of the user of 50% of a maximum protrusion distance of the mandible to set an oxygenation capacity of the user; wherein the upper locking mechanism and the lower locking mechanism are configured to be lockable or unlockable or both by a pressure of a tongue the user or the mandible or both; placing the upper component on at least a portion of the upper teeth; placing the lower component on at least a portion of the lower teeth; and locking the upper locking mechanism and the lower locking mechanism together in a position corresponding to a desired protrusion position to set the oxygenation capacity of the user. In one aspect, the upper locking mechanism and the lower locking mechanism each comprise at least one stub and recess configured to engage each other when a locking pressure is applied with the tongue or the mandible or both; the upper locking mechanism and the lower locking mechanism each comprise at least one stub and recess configured to disengage each other when an unlocking pressure is applied with the tongue or the mandible or both; the upper locking mechanism and the lower locking mechanism are configured to be lockable or unlockable or both during use of the oral appliance; the upper locking mechanism and the lower locking mechanism have at least 3 positions; the upper locking mechanism and the lower locking mechanism are configured to accommodate an underbite or an overbite of the user; or the upper locking mechanism and the lower locking mechanism advance from a centric relation at least 1, 2, 3, 4, 5, 6, 7, 8, 9, or 10 mm. In another aspect, the oral appliance further comprises at least one coupling component that couples the upper component and the lower component. In another aspect, the oral appliance further comprises a strap for attachment to a helmet, facemask, or other face or head protecting device. In another aspect, the at least one position of the lower component relative to the upper component corresponding to at least one protrusion position of the mandible comprises protrusion positions in a range of 0% to 100% of the maximum protrusion distance to allow adjustment of the oxygenation capacity of the user. In another aspect, the oral appliance further comprises at least one of the upper component, the lower component, the upper locking mechanism, or the lower locking mechanism comprises plastic, rubber, or acrylic that can be molded to the upper, lower, or both the upper and lower teeth. In another aspect, the oral appliance is created in a predetermined size. In another aspect, the upper locking mechanism, the lower locking mechanism, or both, is oriented vertically or partially vertically, but not horizontally.

### BRIEF DESCRIPTION OF THE DRAWINGS

For a more complete understanding of the features and advantages of the present invention, reference is now made to the detailed description of the invention along with the accompanying figures, in which:

FIG. 1 shows an exemplary prior art maxilla mouthguard.

FIG. 2 shows an exemplary prior art rubber maxillary and mandibular mouthguard with the mandible behind the maxilla.

FIGS. 3A and 3B show side views of an exemplary oral appliance of the present invention. FIG. 3A shows the oral appliance 300 in an unlocked position, and FIG. 3B shows the oral appliance in a locked position.

FIG. 4 shows a method embodiment according to the present invention.

FIG. 5 shows another embodiment according to the present invention that further includes a strap.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

Athletes frequently exercise to build their shoulders, necks, and chests as part of their sports regimen. Athletes also frequently need maximum oxygenation to have an increased capacity for athletic performance. Athletic mouthguards typically place the teeth in centric relation (CR) or centric occlusion (CO). A mouthguard or oral appliance that increases the user's oxygenation, and that can be easily adjusted by the user, is desirable.

The present invention answers the need for an oral appliance that increases oxygenation in athletes. The present invention includes an oral appliance configured to increase the oxygenation capacity of an athlete by setting his or her mandible at a protrusion position that opens the patient's airway significantly and permits easy adjustment by the athlete to set an increased oxygenation level.

The oral appliance will have 3 or more positions to be locked in, but most often 3 positions. The position will range from Centric Relation, to 10 mm advanced from centric relation. In dentistry, a centric relation is the mandibular jaw position in which the head of the condyle is situated as far anterior and superior as it possibly can within the mandibular fossa/glenoid fossa. The oral appliance is also unique in that it will have a customizable gel liner on part of the appliance that will encase the teeth. The appliance can also have a removable or fixed extra strap for sports requiring helmet, such as football.

As such, the present invention is a sports guard with the mandible in and advanced position. The sports guards of the prior art are in a centric relation.

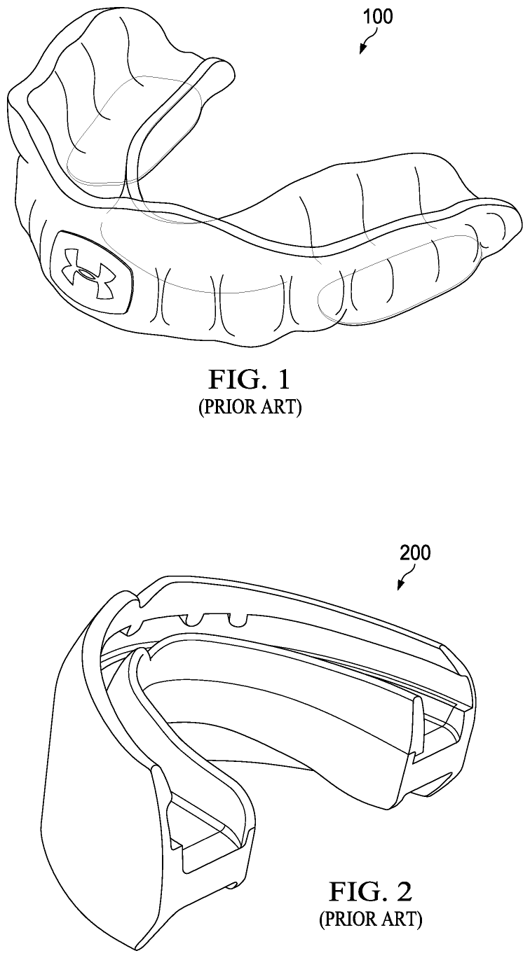

FIG. 1 shows an exemplary prior art maxilla mouthguard 100.

FIG. 2 shows an exemplary prior art rubber maxillary and mandibular mouthguard 200 with the mandible behind the maxilla.

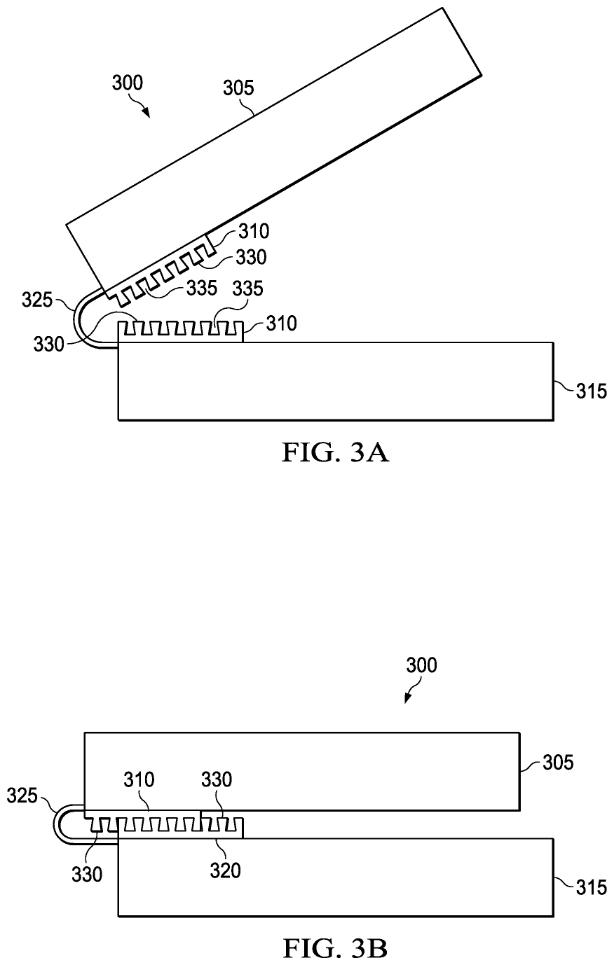

FIGS. 3A and 3B show side views of an exemplary oral appliance 300 according to the present invention. In this embodiment, the oral appliance 300 includes an upper component 305, which in turn includes an upper locking mechanism 310, a lower component 315, which in turn includes a lower locking mechanism 320, and at least one optional coupling component 325, positioned at a posterior end of each of the upper component 305 and the lower component 315, that couples the upper component 305 and the lower component 315. FIG. 3A shows the oral appliance 300 in an unlocked position, and FIG. 3B shows the oral appliance in a locked position.

The upper component 305 is configured to fit onto at least a portion of a user's upper teeth (not shown), and the lower component 315 is configured to fit onto at least a portion of the user's lower teeth (not shown). The lower locking mechanism 320 is configured to engage the upper locking mechanism 310 in at least one position of the lower component 315 relative to the upper component 305 that corresponds to at least one protrusion position of the user's mandible of 50% of a maximum protrusion distance to set an oxygenation capacity of the user. Herein, 0% of a maximum protrusion distance is defined as the position of the mandible at rest, and 100% of a maximum protrusion distance is defined as the maximum distance that the mandible can be protruded forward from the rest position. The 100% maximum mandible protrusion varies among people, but in adults it is typically about 10 mm. Thus, 50% of a maximum protrusion distance is 50% the maximum distance that the mandible can be protruded forward from the rest position, typically 5 mm in adults. The upper locking mechanism 310 and the lower locking mechanism 320 are configured to be lockable or unlockable or both by a pressure of the user's tongue or the user's mandible or both (not shown). The upper locking mechanism 310 and the lower locking mechanism may take a variety of forms. In one embodiment, one of the locking mechanisms includes at least one stub 330, e.g. a short protrusion, that is configured to engage a recess 335 on the other locking mechanism with the application of pressure from the user's tongue or the user's mandible or both. The stub 330 and the recess 335 are each configured to disengage when an unlocking pressure is applied with the user's tongue or the user's mandible or both. In that way, an athlete can increase or decrease her oxygenation capacity while performing athletically without removing the oral appliance 300.

In one embodiment, the oral appliance 300 has a upper locking mechanism 310 and a lower locking mechanism 320 configured to permit locking the upper component 305 and the lower component 315 in positions that correspond to mandible protrusion positions of 0% to 100% of maximum protrusion of the mandible to permit a wide degree of adjustment of oxygenation capacity for different users. The oral appliance may include a range of locking positions that also accommodate a user's underbite or overbite.

In one embodiment, the upper locking mechanism 310, the lower locking mechanism 320, both is oriented vertically or at an angle to vertical, but not horizontally.

Any constituent portion of the oral appliance 300, including the upper component 305, the upper locking mechanism 310, the lower component 315, the lower locking mechanism 320, the coupling component 325, or stubs 330 may include plastic, rubber, or acrylic.

The oral appliance 300 may be made in a number of predetermined sized, e.g., small (S), medium (M), large (L), XL (extra-large), or XXL (extra-extra-large), the exact dimensions of each predetermined size to be determined by average sizes of human mouths, to economize in manufacturing while accommodating differently sized users.

The oral appliance may be included in a kit, which may also include instructions and one or more implements or supplies commonly used in the fitting or installation of an oral appliance in a user's mouth.

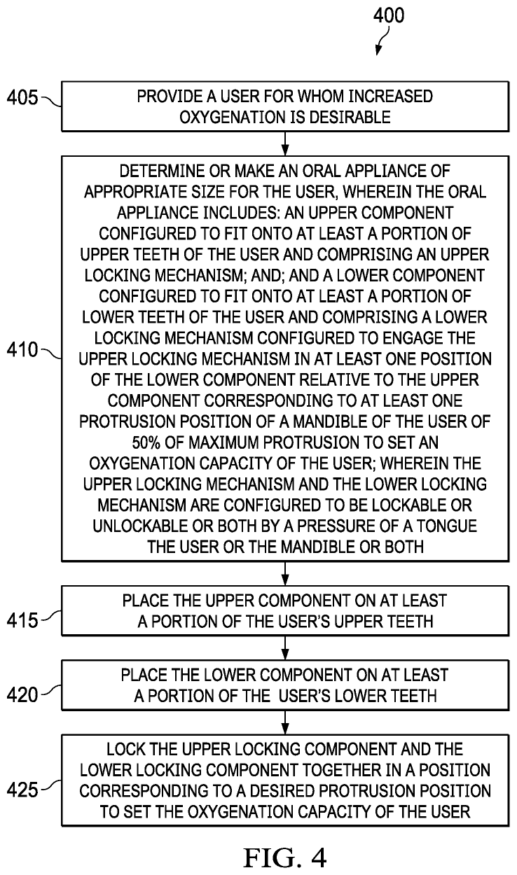

FIG. 4 shows a method embodiment 400 of the present invention. Block 405 includes providing a user for whom increased oxygenation is desirable. Block 410 includes determining or making an oral appliance of appropriate size for the user, wherein the oral appliance includes an upper component configured to fit onto at least a portion of upper teeth of the user and including an upper locking mechanism; and a lower component configured to fit onto at least a portion of lower teeth of the user and including a lower locking mechanism configured to engage the upper locking mechanism in at least one position of the lower component relative to the upper component corresponding to at least one protrusion position of a mandible of the user of 50% of maximum protrusion of the mandible to set an oxygenation capacity of the user; wherein the upper locking mechanism and the lower locking mechanism are configured to be lockable or unlockable or both by a pressure of a tongue the user or the mandible or both.

Placing the upper component on at least a portion of the user's upper teeth is included in Block 415, while placing the lower component on at least a portion of the user's lower teeth in included in Block 420. Block 425 includes locking the upper locking component and the lower locking mechanism together in a position corresponding to a desired protrusion position to set the oxygenation capacity of the user.

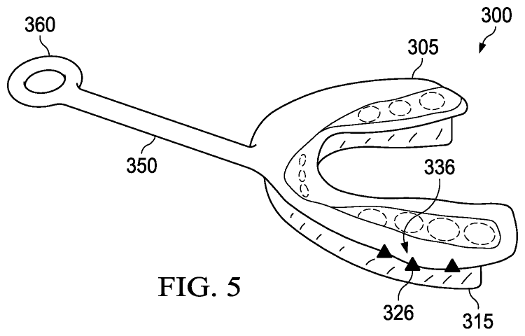

FIG. 5 shows another embodiment of a mouthguard of the present invention that also includes upper component 305 is configured to fit onto at least a portion of a user's upper teeth (not shown), and the lower component 315 is configured to fit onto at least a portion of the user's lower teeth (not shown). The lower locking mechanism 326 is configured to engage the upper locking mechanism 336 in at least one position of the lower component 315 relative to the upper component 305 that corresponds to at least one protrusion position of the user's mandible of 50% of a maximum protrusion distance to set an oxygenation capacity of the user. In this embodiment, the lower locking mechanism 326 is shown as having three locking positions (not to scale), which of course can be reversed, that is, the upper locking mechanism 336 can lock into the lower locking mechanism 326. Herein, 0% of a maximum protrusion distance is defined as the position of the mandible at rest, and 100% of a maximum protrusion distance is defined as the maximum distance that the mandible can be protruded forward from the rest position. The 100% maximum mandible protrusion varies among people, but in adults it is typically about 10 mm. Thus, the upper component 305 and the lower component 315 will generally move from a centric relation, to 1, 2, 3, 4, 5, 6, 7, 8, 9, or 10 mm advanced from the centric relation. Thus, 50% of a maximum protrusion distance is 50% the maximum distance that the mandible can be protruded forward from the rest position, typically 5 mm in adults. The upper locking mechanism 336 and the lower locking mechanism 326 are configured to be lockable or unlockable or both by a pressure of the user's tongue or the user's mandible or both (not shown), and can even be adjusted during use. The upper locking mechanism 336 and the lower locking mechanism may take a variety of forms. In one embodiment, one of the locking mechanisms includes at least one stub, e.g. a short protrusion, that is configured to engage a recess on the other locking mechanism with the application of pressure from the user's tongue or the user's mandible or both. The stub and the recess are each configured to disengage when an unlocking pressure is applied with the user's tongue or the user's mandible or both. In that way, an athlete can increase or decrease her oxygenation capacity while performing athletically without removing the oral appliance 300. The oral appliance 300 further comprises a strap 350, depicted in this embodiment with a loop 360 that allows for the oral appliance 300 to be attached to, e.g., a helmet, a facemask, or other head protection device. In this embodiment, the strap 350 is integral with the upper component 205, however, the present invention also include that the strap can be integral with the lower component 315, or can include an orifice into which a strap or tether can be attached and/or removed.

In one embodiment, the oral appliance 300 has a upper locking mechanism 336 and a lower locking mechanism 326 configured to permit locking the upper component 305 and the lower component 315 in positions that correspond to mandible protrusion positions of 0% to 100% of maximum protrusion of the mandible to permit a wide degree of adjustment of oxygenation capacity for different users. The oral appliance may include a range of locking positions that also accommodate a user's underbite or overbite. Each of the upper component 305 and the lower component 315 can include customizable gel liner on part of the appliance that will encase the teeth, and the entire device will, generally, not include any solid surfaces in contact with the inside of the mouth, but rather, will be elastomeric. Thus, the upper component 305 and the lower component 315 will generally move from a centric relation, to 1, 2, 3, 4, 5, 6, 7, 8, 9, or 10 mm advanced from the centric relation.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step, or a limitation) or group of integers (e.g., feature(s), element(s), characteristic(s), property(ies), method/process(s) steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the devices and/or methods disclosed and claimed herein can be made and executed without undue experimentation in light of the present disclosure. While the devices and/or methods of this invention have been described in terms of particular embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

Furthermore, no limitations are intended to the details of construction or design herein shown, other than as described in the claims below. It is therefore evident that the particular embodiments disclosed above may be altered or modified and all such variations are considered within the scope and spirit of the disclosure. Accordingly, the protection sought herein is as set forth in the claims below.

Modifications, additions, or omissions may be made to the systems and apparatuses described herein without departing from the scope of the invention. The components of the systems and apparatuses may be integrated or separated. Moreover, the operations of the systems and apparatuses may be performed by more, fewer, or other components. The methods may include more, fewer, or other steps. Additionally, steps may be performed in any suitable order.

To aid the Patent Office, and any readers of any patent issued on this application in interpreting the claims appended hereto, applicants wish to note that they do not intend any of the appended claims to invoke 35 U.S.C. § 112(f) as it exists on the date of filing hereof unless the words “means for” or “step for” are explicitly used in the particular claim.

What is claimed is:

1. An oral appliance for increasing oxygenation of an athlete comprising:
an upper component configured to fit onto at least a portion of upper teeth of a user and comprising an upper locking mechanism; and
a lower component configured to fit onto at least a portion of lower teeth of the user and comprising a lower locking mechanism configured to engage the upper locking mechanism in at least one position of the lower component relative to the upper component corresponding to at least one protrusion position of a mandible of the user of 50% of a maximum protrusion distance to set an oxygenation capacity of the user;
wherein the upper locking mechanism and the lower locking mechanism are configured to be lockable or unlockable or both by a pressure of a tongue the user or the mandible or both.

2. The oral appliance of claim 1, wherein at least one of:
the upper locking mechanism and the lower locking mechanism each comprise at least one stub and recess configured to engage each other when a locking pressure is applied with the tongue or the mandible or both;
the upper locking mechanism and the lower locking mechanism each comprise at least one stub and recess configured to disengage each other when an unlocking pressure is applied with the tongue or the mandible or both;
the upper locking mechanism and the lower locking mechanism are configured to be lockable or unlockable or both during use of the oral appliance;
the upper locking mechanism and the lower locking mechanism have at least 3 positions;
the upper locking mechanism and the lower locking mechanism are configured to accommodate an underbite or an overbite of the user; or
the upper locking mechanism and the lower locking mechanism advance from a centric relation at least 1, 2, 3, 4, 5, 6, 7, 8, 9, or 10 mm.

3. The oral appliance of claim 1, further comprising at least one coupling component that couples the upper component and the lower component.

4. The oral appliance of claim 1, further comprising a strap for attachment to a helmet, facemask, or other face or head protecting device.

5. The oral appliance of claim 1, wherein the at least one position of the lower component relative to the upper component corresponding to at least one protrusion position of the mandible comprises protrusion positions in a range of 0% to 100% of the maximum protrusion distance to allow adjustment of the oxygenation capacity of the user.

6. The oral appliance of claim 1, wherein at least one of the upper component, the lower component, the upper locking mechanism, or the lower locking mechanism comprises plastic, rubber, or acrylic that can be molded to the upper, lower, or both the upper and lower teeth.

7. The oral appliance of claim 1, wherein the oral appliance is created in a predetermined size.

8. The oral appliance of claim 1, wherein the upper locking mechanism, the lower locking mechanism, or both, is oriented vertically or partially vertically, but not horizontally.

9. A kit comprising:
at least one oral appliance for increasing oxygenation of an athlete comprising:
an upper component configured to fit onto at least a portion of upper teeth of a user and comprising an upper locking mechanism; and
a lower component configured to fit onto at least a portion of lower teeth of the user and comprising a lower locking mechanism configured to engage the upper locking mechanism in at least one position of the lower component relative to the upper component corresponding to at least one protrusion position of a mandible of the user of 50% of a maximum protrusion distance to set an oxygenation capacity of the user;
wherein the upper locking mechanism and the lower locking mechanism are configured to be lockable or unlockable or both by a pressure of a tongue the user or the mandible or both.

10. The kit of claim 9, wherein at least one of:
the upper locking mechanism and the lower locking mechanism each comprise at least one stub and recess configured to engage each other when a locking pressure is applied with the tongue or the mandible or both;
the upper locking mechanism and the lower locking mechanism each comprise at least one stub and recess configured to disengage each other when an unlocking pressure is applied with the tongue or the mandible or both;
the upper locking mechanism and the lower locking mechanism are configured to be lockable or unlockable or both during use of the oral appliance;
the upper locking mechanism and the lower locking mechanism have at least 3 positions;
the upper locking mechanism and the lower locking mechanism are configured to accommodate an underbite or an overbite of the user; or
the upper locking mechanism and the lower locking mechanism advance from a centric relation at least 1, 2, 3, 4, 5, 6, 7, 8, 9, or 10 mm.

11. The kit of claim 9, wherein the at least one position of the lower component relative to the upper component corresponding to at least one protrusion position of the mandible comprises protrusion positions in a range of 0% to 100% of the maximum protrusion distance to allow adjustment of the oxygenation capacity of the user.

12. The kit of claim 9, further comprising a strap for attachment to a helmet, facemask or other face or head protecting device.

13. The kit of claim 9, wherein at least one of the upper component, the lower component, the upper locking mechanism, or the lower locking mechanism comprises plastic, rubber, or acrylic that can be molded to the upper, lower, or both the upper and lower teeth.

14. The kit of claim 9, wherein the oral appliance is created in a predetermined size.

15. A method of increasing oxygenation comprising:
providing a user for whom increased oxygenation is desirable;
determining or making an oral appliance of appropriate size for the user, wherein the oral appliance comprises:
an upper component configured to fit onto at least a portion of upper teeth of the user and comprising an upper locking mechanism; and
a lower component configured to fit onto at least a portion of lower teeth of the user and comprising a lower locking mechanism configured to engage the upper locking mechanism in at least one position of the lower component relative to the upper component corresponding to at least one protrusion position of a mandible of the user of 50% of a maximum protrusion distance to set an oxygenation capacity of the user;
wherein the upper locking mechanism and the lower locking mechanism are configured to be lockable or unlockable or both by a pressure of a tongue the user or the mandible or both;
placing the upper component on at least a portion of the upper teeth;
placing the lower component on at least a portion of the lower teeth; and
locking the upper locking mechanism and the lower locking mechanism together in a position corresponding to a desired protrusion position to set the oxygenation capacity of the user.

16. The method of claim 15, wherein at least one of:
the upper locking mechanism and the lower locking mechanism each comprise at least one stub and recess configured to engage each other when a locking pressure is applied with the tongue or the mandible or both;
the upper locking mechanism and the lower locking mechanism each comprise at least one stub and recess configured to disengage each other when an unlocking pressure is applied with the tongue or the mandible or both;
the upper locking mechanism and the lower locking mechanism are configured to be lockable or unlockable or both during use of the oral appliance;
the upper locking mechanism and the lower locking mechanism have at least 3 positions;
the upper locking mechanism and the lower locking mechanism are configured to accommodate an underbite or an overbite of the user;
the upper locking mechanism and the lower locking mechanism advance from a centric relation at least 1, 2, 3, 4, 5, 6, 7, 8, 9, or 10 mm; or the oral appliance is created in a predetermined size.

17. The method of claim 15, wherein the oral appliance further comprises at least one coupling component that couples the upper component and the lower component.

18. The method of claim 15, wherein the at least one position of the lower component relative to the upper component corresponding to at least one protrusion position of the mandible comprises protrusion positions in a range of 0% to 100% of the maximum protrusion distance to allow adjustment of the oxygenation capacity of the user.

19. The method of claim 15, wherein at least one of the upper component, the lower component, the upper locking mechanism, or the lower locking mechanism comprises plastic, rubber, or acrylic that can be molded to the upper, lower, or both the upper and lower teeth.

20. The method of claim 15, further comprising a strap for attachment to a helmet, facemask or other face or head protecting device.
